(19) United States
(12) Patent Application Publication
(10) Pub. No.: US 2010/0273736 A1
(43) Pub. Date: Oct. 28, 2010

(54) COSMETIC COMPOSITION COMPRISING GLYCOGEN FOR SKIN APPLICATION WITH VELVET EFFECT

(75) Inventors: Leonardo Marchitto, Porto Recanati (IT); Lorella Ragni, Chiaravalle (IT); Francesca Mariotti, Pesaro (IT)

Correspondence Address: OBLON, SPIVAK, MCCLELLAND MAIER & NEUSTADT, L.L.P., 1940 DUKE STREET, ALEXANDRIA, VA, 22314, US

(73) Assignee: Aziende Chim. Riun. Ang. Franc. A.C.R.A.F. S.P.A., Roma (IT)

(21) Appl. No.: 12/746,878

(22) PCT Filed: Jan. 21, 2009
(86) PCT No.: PCT/EP2009/050632

§ 371 Date: Jun. 8, 2010

(30) Foreign Application Priority Data
Feb. 1, 2008 (EP) 08425060.4

(51) Int. Cl. A61K 8/73; A61Q 19/00

(52) U.S. Cl. 514/54

(57) ABSTRACT

This invention relates to a cosmetic composition for skin application having a velvet effect on the skin comprising, as the cosmetic active ingredient, a glycogen having less than 1,000 ppm of nitrogen and less than 1% by weight of reducing sugars, in at least one cosmetically-acceptable vehicle.

### FIELD OF THE INVENTION

This invention relates to a cosmetic composition for skin application having a velvet effect and comprising glycogen as the cosmetic active ingredient. In addition to this, this invention relates to a new cosmetic use of glycogen as a velvet agent in a cosmetic composition for skin application. Finally, this invention relates to a new cosmetic use of glycogen for the preparation of a cosmetic composition having a velvet effect for skin application.

### STATE OF THE ART

Glycogen is a polysaccharide of animal origin mainly comprised of molecules of D-glucose linked together by α-1-4 glucoside bonds with branches every five-ten glucose units comprising glucoside α-1-6 bonds. The frequency and level of branching of the glycogen varies in relation to the animal species from which it has been obtained. The molecular weight of natural glycogen is of the order of 10⁶-10⁷ Dalton. In nature glycogen is always bound to a protein, glycogenin, an enzyme correlated with the process of cell glycogen synthesis.

Glycogen extracted from natural sources is used in the cosmetics industry as an emollient and hydrating agent, as described in JP-A-62-178 505 and JP-A-63-290 809, as an anti-ageing agent as described in U.S. Pat. No. 5,093,109 and JP-A-2003-335651, and as a humectant and lubricant in ophthalmic solutions as described in WO99/47120.

The quality of commercial glycogen depends on the presence in greater or lesser quantities of protein residues (measured in terms of quantity of nitrogen, expressed as ppm) and reducing sugars.

Patent EP 654,048 describes a high-quality glycogen having a very low nitrogen content (<60 ppm) and reducing sugars content (<0.25% by weight).

### DEFINITIONS

For the purposes of this description and the following claims, by the term “velvet” or “velvet effect” is meant the achievement of a sensory effect of a soft and smooth skin.

### SUMMARY OF THE INVENTION

The Applicant has surprisingly found that the use of glycogen in a cosmetic formulation for skin application imparts a velvet effect to the skin.

Thus, in a first aspect this invention relates to a cosmetic composition for skin application having a velvet effect on the skin, comprising as the cosmetic active ingredient a glycogen containing less than 1000 ppm of nitrogen and less than 1% by weight of reducing sugars, in at least one cosmetically-acceptable vehicle.

In a second aspect this invention relates to the cosmetic use of glycogen, preferably having a low nitrogen and reducing sugars content, as a velvet agent in a cosmetic composition for skin application.

In a third aspect this invention relates to the cosmetic use of glycogen, preferably having a low nitrogen and reducing sugars content, for the preparation of a cosmetic composition for skin application, the said cosmetic composition having a velvet effect on the skin.

The Applicant has found that application of the cosmetic composition according to this invention to the skin makes it possible to achieve a velvet effect on the treated skin.

In particular, the velvet effect is manifested through a sensation of softness and smoothness perceived by the treated individual about three minutes after application of the cosmetic composition according to this invention.

The Applicant has also observed that the cosmetic composition according to this invention is rapidly absorbed by the skin and provides a lifting effect of firm and smooth skin.

Moreover, the Applicant has also observed that the cosmetic composition according to this invention imparts greater brightness and lustre to treated skin.

Finally, the Applicant has observed that the cosmetic composition according to this invention can be easily manufactured in industrial applications and is stable over time.

### BRIEF DESCRIPTION OF THE FIGURES

FIG. 1 shows graphs comparing evaluations of the products in Example 1.

FIG. 2 shows graphs comparing evaluations of the products in Example 2.

FIG. 3 shows graphs comparing evaluations of the products in Example 3.

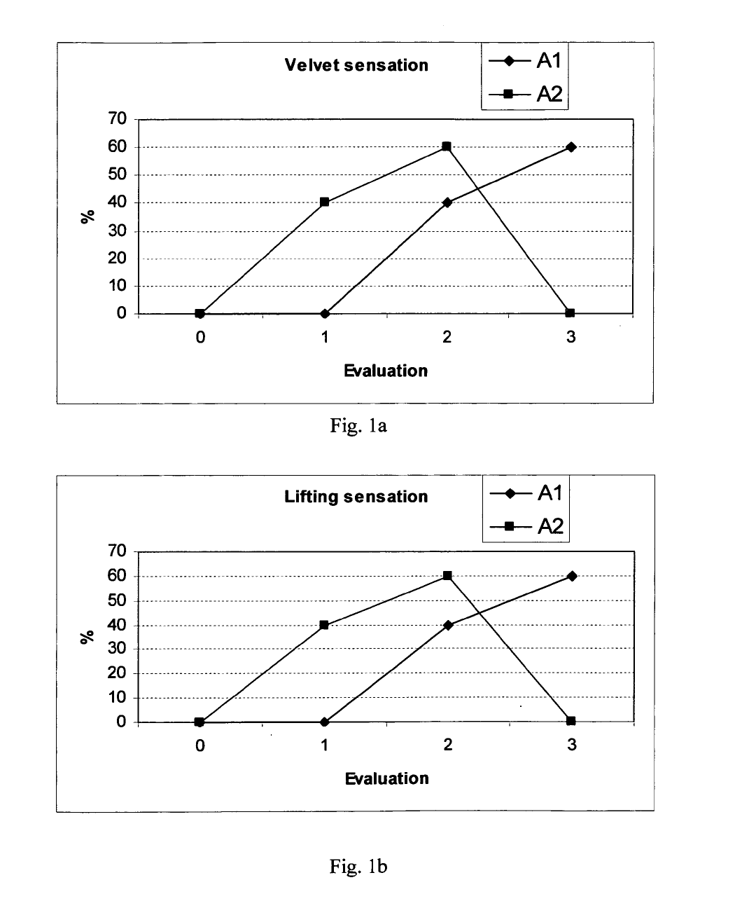

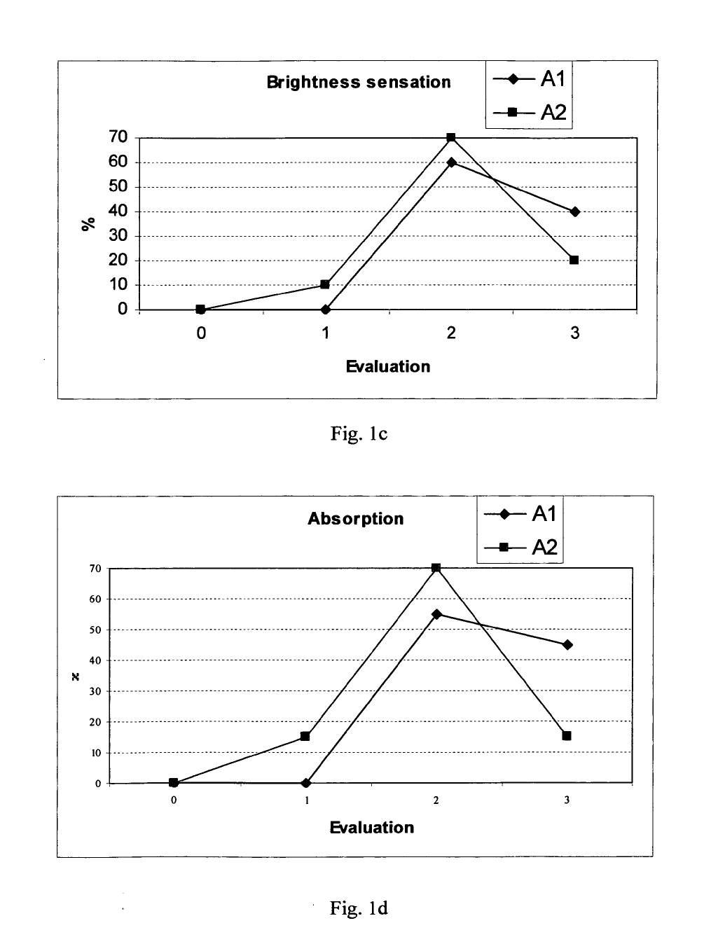

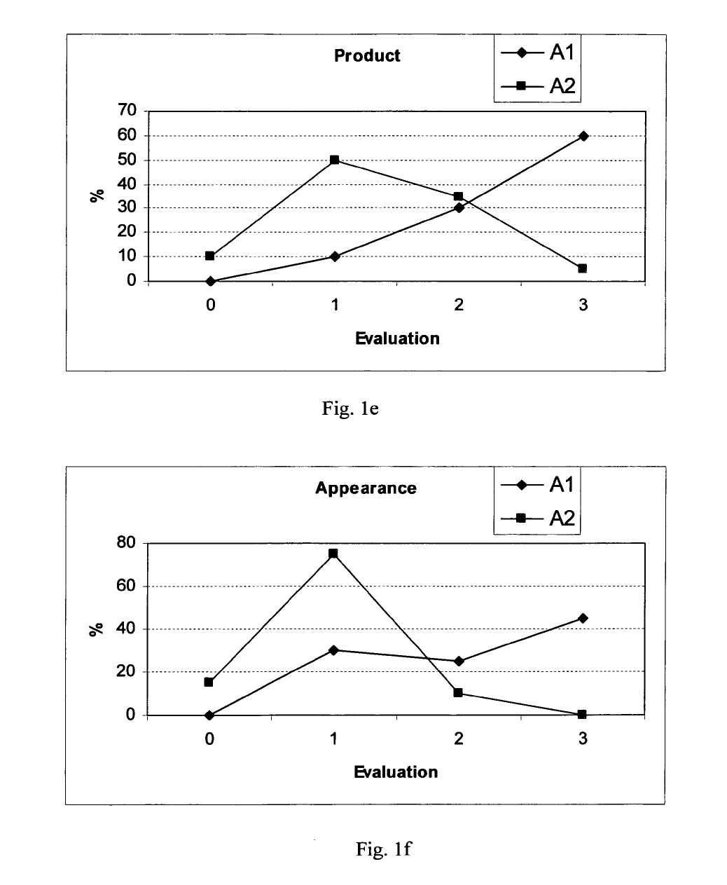

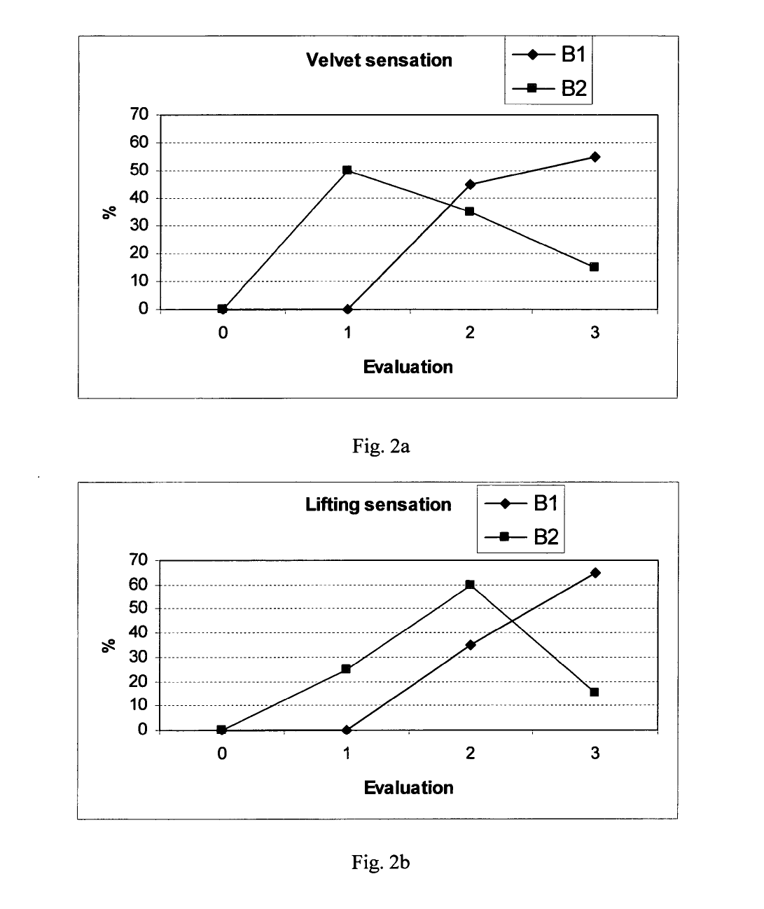

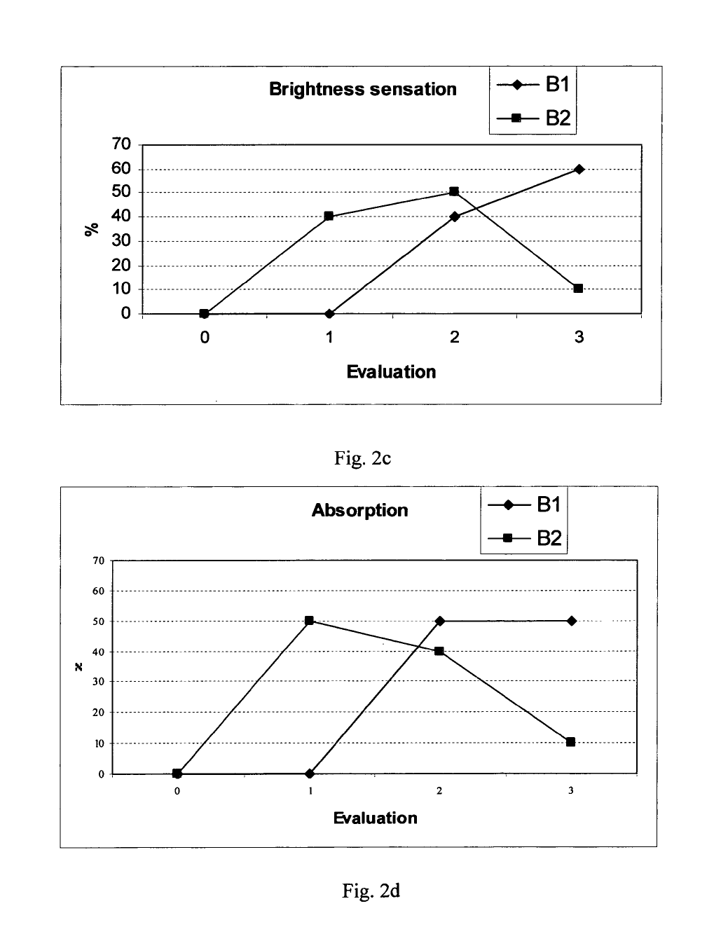

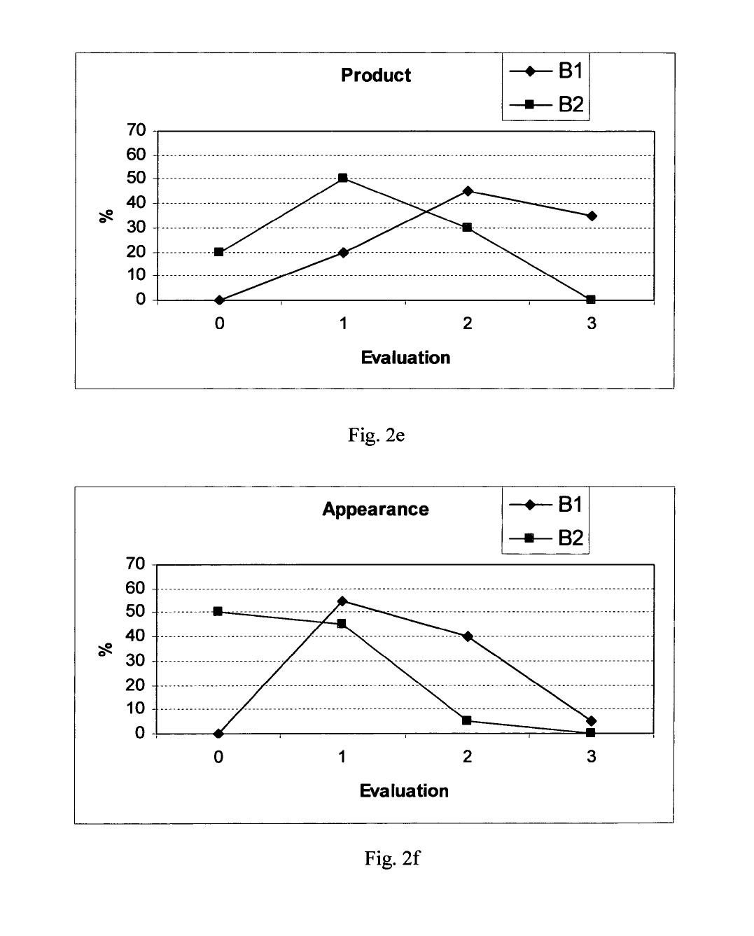

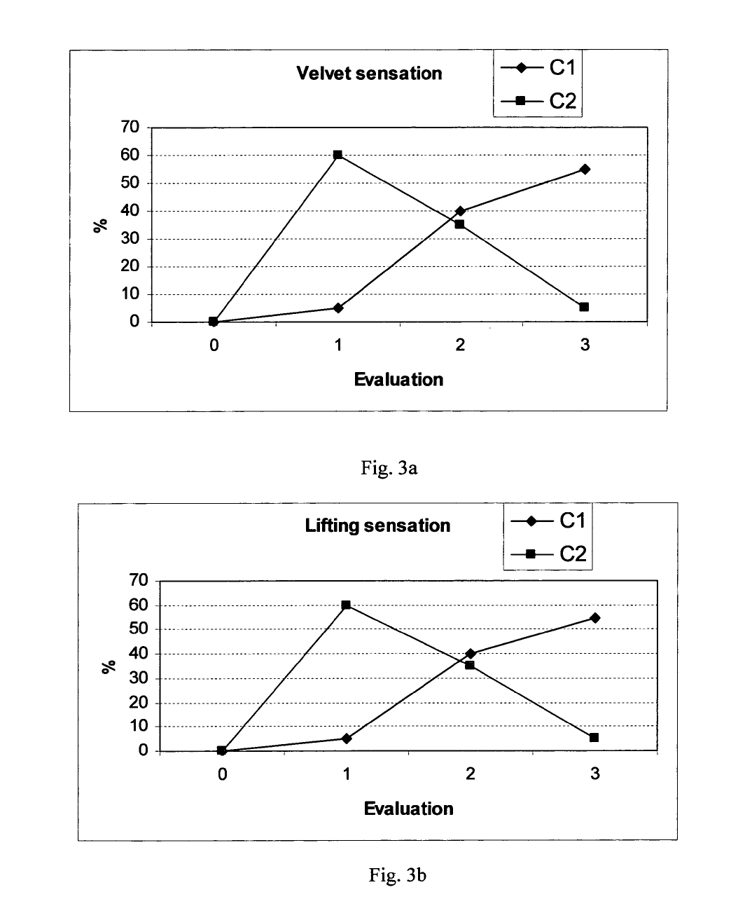

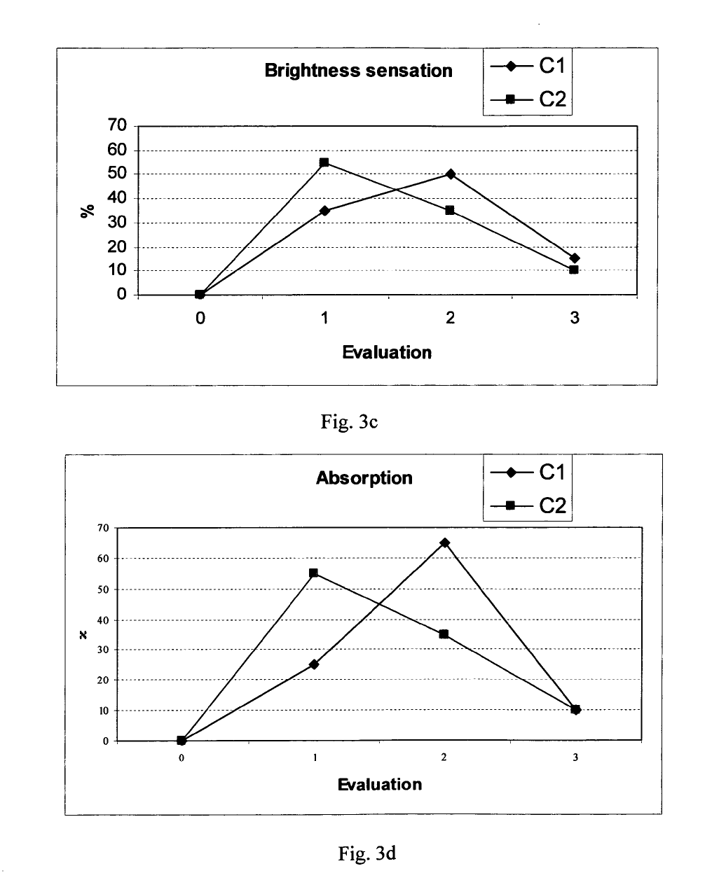

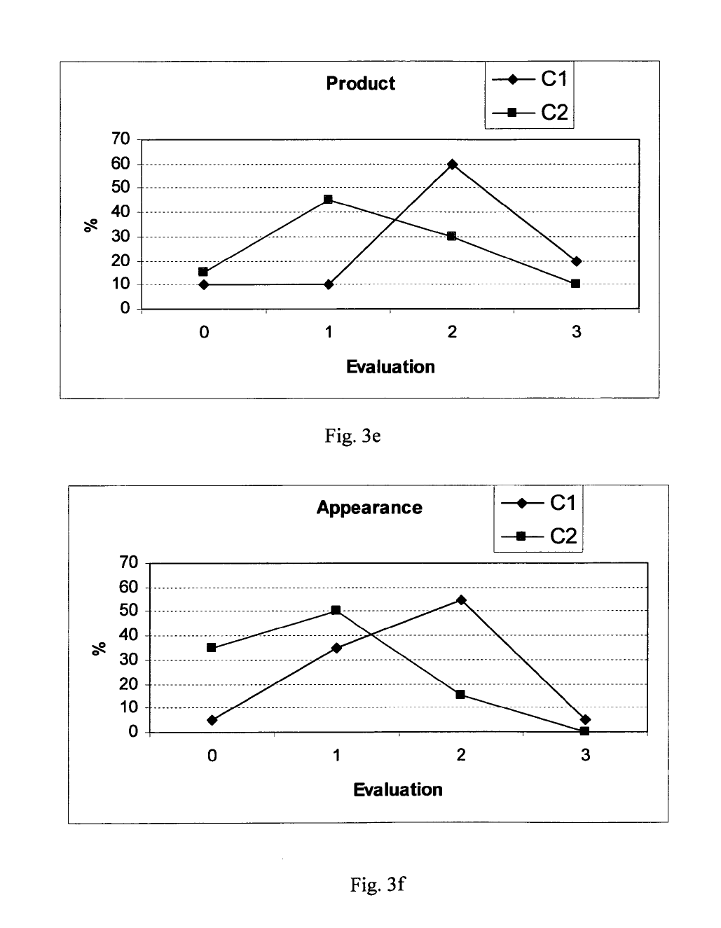

### DETAILED DESCRIPTION OF THE INVENTION

The glycogen used in this invention is obtained from natural glycogen which may be extracted from animals or fungi. Molluscs, in particular mussels (Mytilus edulis and Mytilus gallus provincialis) are a particularly useful source of glycogen because they are available in large quantities at low cost and contain a reasonable quantity of glycogen (on average between 2.5% and 3.9% by weight). Other natural sources of glycogen include other bivalve molluscs such as clams, oysters, some species of gastropods or sea snails, such as slipper limpets (Crepidula fornicata), as well as organs of vertebrate animals which are rich in glycogen such as the liver and muscles.

The glycogen used in this invention may be used as such as obtained from extraction processes or may be treated in subsequent purification procedures. As already mentioned, the quality of a commercial glycogen depends on the presence of a greater or lesser quantity of protein residues (measured in terms of quantity of nitrogen expressed as ppm) and reducing sugars.

For the purposes of this invention the use of a glycogen having a low content of reducing sugars and nitrogen is preferred. Examples of commercial products preferably used in this invention are glycogens produced and distributed by Sigma-Aldrich.

Preferably, the glycogen used in this invention comprises less than 1% by weight, and more preferably less than 0.25% by weight of reducing sugars, measured in accordance with the method by F. D. Snell and Snell, “Colorimetric Methods of Analysis”, New York, 1954, vol. III, p. 204.

Preferably, the glycogen used in this invention comprises less than 1000 and more preferably less than 100 ppm of nitrogen measured using the Kjeldahl method.

Advantageously, the glycogen used in this invention is Polglumyt™ glycogen, the trade name of a deproteinated glycogen produced and distributed by A.C.R.A.F. S.p.A., Rome, Italy, and obtained in accordance with the purification procedure described in patent EP 654048 B1.

Preferably, the cosmetic composition according to this invention comprises a quantity of glycogen of between about 0.1% and about 15%, more preferably between 0.5% and 10%, and even more preferably between 1% and 5% by weight relative to the weight of the total formulation.

The cosmetic composition according to this invention comprises liquid or semi-solid formulations.

The liquid formulations for cosmetic use according to this invention comprise solutions, emulsions, microemulsions, lotions, foams, milks, oils, relaxants or suspensions of widely varying viscosity.

The liquid formulations may for example be aqueous solutions, hydroalcoholic solutions, oily solutions, emulsions obtained by dispersing an oily phase in an aqueous phase (oil-in-water) or vice-versa an aqueous phase in an oily phase (water-in-oil), and suspensions obtained by dispersing a dispersed phase comprising solid particles in a dispersing medium generally represented by an aqueous or oily liquid having a particular viscosity.

The semi-solid formulations for cosmetic use according to this invention comprise creams, gels, ointments, pastes, cream-gels, sticks and waxes.

The formulations for cosmetic use of this invention may comprise various cosmetically-acceptable additives or vehicles which are useful in the preparation of cosmetic products and known to those skilled in the art such as, for example, emulsifiers, hydrating agents, solvents, emollients, stabilisers, viscosity agents, preservatives, lubricants, sequestrating or chelating agents, fillers, fragrances, perfumes, absorbents, colouring agents and opacifiers, antioxidants, plant extracts and oils, vitamins, protective substances, essential oils, keratin-active substances and amino acids.

The liquid formulations for cosmetic use according to this invention preferably comprise at least one solvent, at least one hydrating agent, at least one sequestering agent, and at least one preservative.

The semi-solid formulations for cosmetic use according to this invention preferably comprise at least one solvent, at least one emulsifier, at least one viscosity agent, at least one hydrating agent, at least one emollient, and at least one preservative.

Suitable solvent additives comprise, for example, water, alcohols, ketones (such as acetone and methylisobutyl ketone), glycols (such as ethylene glycol, propylene glycol and butylene glycol), polyethylene glycols (such as PEG-40, PEG-50, PEG-60), alkyl acetates (such as amyl acetate, isopropyl acetate, butyl acetate), paraffins and isoparaffins, cycloalkyls (such as cyclohexane), glycerine, natural and synthetic oils, natural and synthetic triglycerides.

Depending upon the solvent used we can make a distinction between aqueous formulations and non-aqueous or water-free formulations.

In aqueous formulations the water represents the main component of the cosmetic composition and can even amount to a quantity of up to 99% by weight relative to the weight of the total formulation. Aqueous formulations contain a quantity of water of preferably between 25% and 95%, preferably between 50% and 90% by weight relative to the weight of the total formulation.

The aqueous formulations of the cosmetic composition according to this invention may preferably comprise a total quantity of non-aqueous solvents of between about 0.1% and about 60%, more preferably between 1% and 40%, and even more preferably between 5% and 35% by weight relative to the weight of the total formulation.

In non-aqueous or water-free formulations, water is absent and the total quantity of solvent additives other than water is between about 1% and about 99%, preferably between 25% and 95%, preferably between 50% and 90% by weight relative to the weight of the total formulation. The expression “water is absent” means that the quantity of water is less than 0.01% by weight relative to the weight of the total formulation.

Suitable emulsifying additives may be non-ionic, cationic, anionic and amphoteric surfactants, or combinations thereof. Useful examples of emulsifiers are, for example, sorbitan, long-chain ethoxylated alcohols, alkyl polyglycosides, soaps, alkyl sulfates, such as for example sodium cetyl stearyl sulfate, monoalkyl and dialkyl phosphates, alkyl sulfonates, hydrogenated castor oil, acyl isothionates, saccharose esters, betain, lecithin, quaternary ammonium salts, alkyl oleates, glycerides such as for example caprylocaproyl polyoxylglycerides (caprylocaproyl macrogolglycerides) and emulsifying agents from olive oil.

The cosmetic composition according to this invention preferably comprises a total quantity of emulsifiers of between about 0.1% and about 60%, more preferably between 0.5% and 25%, and even more preferably between 0.5% and 10% by weight relative to the weight of the total formulation.

Useful viscosity agents are represented for example by xanthan gum, hydroxypropylcellulose, hydroxyethylcellulose, carbopol, carrageenans, poloxamers and acacia gum.

The cosmetic composition according to this invention preferably comprises a total quantity of viscosity agents of between about 0.1% and about 25%, more preferably between 0.5% and 10%, and even more preferably between 0.5% and 5% by weight relative to the weight of the total formulation.

Additives having a hydrating action are for example urea, allantoin, hyaluronic acid and its derivates, glycerine, amino acids, acetyl monoethanolamide, butoxypropanol, butyl glycol, low molecular weight polyethylene glycols (such as PEG-40, PEG-50, PEG-60), aloe, mallow, and sorbitol.

The cosmetic composition according to this invention preferably comprises a total quantity of hydrating agents of between about 0.05% and about 25%, more preferably between 0.5% and 10%, and even more preferably between 0.1% and 5% by weight relative to the weight of the total formulation.

Suitable emollient additives include, for example, lanolin, almond oil, olive oil, hydrogenated castor oil, microcrystalline wax, polydimethylsiloxane (dimethicone), polymethylphenylsiloxane, polymers of glycol and silicone, mineral oils, paraffin, ozokerite, ceresine, triglyceride esters, monoglyceride acetylates, ethoxylated glycerides, alkyl esters of fatty acids, fatty acids, long-chain alcohols, sterols, beeswax, polyhydric alcohols, polyesters, and fatty acid amides.

The cosmetic composition according to this invention preferably comprises a total quantity of emollients of between about 0.1% and about 25%, more preferably between 0.5% and 10%, and even more preferably between 0.5% and 5% by weight relative to the weight of the total formulation.

Suitable preservative additives include, for example, alcohols such as ethanol, phenoxyethanol and benzyl alcohol, methyl and propyl parahydroxybenzoate, hydroxyanisole butylate (BHA), sorbates, urea derivates and isothiazolinones.

The cosmetic composition according to this invention preferably comprises a total quantity of preservatives of between about 0.01% and about 2.00%, more preferably between 0.05% and 1.00%, and even more preferably between 0.1% and 0.5% by weight relative to the weight of the total formulation.

Sequestrating or chelating additives include EDTA, HEDTA, alkyl oxalates, lithium or potassium oxalate, sodium or potassium pyrophosphate. The cosmetic composition according to this invention preferably comprises a total quantity of sequestrating or chelating agents of between about 0.01% and about 20%, more preferably between 0.05% and 10%, and even more preferably between 0.1% and 5% by weight relative to the weight of the total formulation.

Suitable stabilising additives are, for example, long-chain alcohols (such as cetyl alcohol, stearyl alcohol) and mixtures thereof, high molecular weight polyethylene glycols (such as PEG-9000 and PEG-14000) and polyvinyl pyrrolidones (such as povidone).

The cosmetic composition according to this invention preferably comprises a total quantity of stabilising agents of between about 0.1% and about 25%, more preferably between 0.5% and 15%, and even more preferably between 1% and 10% by weight relative to the weight of the total formulation.

Opacifiers are for example zinc or aluminium oxide, titanium or zinc dioxide, alumina, mica, aluminium salts of fatty acids, and chalk.

The colouring agents preferably used are readily washable water-soluble colouring agents which do not stain the skin or leave residues such as, for example, Acid Blue 3 C.I. 42051, Acid Blue 9 C.I. 42090, Acid Blue 74 C.I. 73015, Pigment Blue 15 C.I. 74160, Acid Yellow 3 C.I. 47005, Food Grade Yellow 3 C.I. 15985, Acid Yellow 23 C.I. 19140, Acid Yellow 73 C.I. 45350, Acid Red 14 C.I. 14720, Acid Red 18 C.I. 16255, Acid Red 27 C.I. 16185, Acid Red 51 C.I. 45430, Acid Green 1 C.I. 10020, Acid Green 25 C.I. 61570, and mixtures thereof.

The cosmetic composition according to this invention preferably comprises a total quantity of opacifiers and colouring agents of between about 0.01% and about 15%, more preferably between 0.05% and 5% by weight relative to the weight of the total formulation.

The following examples serve to illustrate the invention without however limiting it.

### Example 1

Two body cream formulations were prepared using the ingredients in Table 1 below. The quantities stated in Table 1 are percentages by weight (g/100 g).

TABLE 1

| | A1 (invention) | A2 (comparison) |
|---|---|---|
| Polglumyt ™ | 2 | — |
| Methyl p-hydroxybenzoate | 0.18 | 0.18 |
| Propyl p-hydroxybenzoate | 0.02 | 0.02 |
| Lanette O (cetyl stearyl alcohol) | 3 | 3 |
| Lanette E (sodium cetyl stearyl | 0.5 | 0.5 |
| sulfate) | | |
| Dimethicone 300 cps | 0.5 | 0.5 |
| Allantoin | 0.2 | 0.2 |
| Purified water q.s. | 100 | 100 |

### Example 2

Two face cream formulations were prepared using the ingredients in Table 2 below. The quantities stated in Table 2 are percentages by weight (g/100 g).

TABLE 2

| | B1 (invention) | B2 (comparison) |
|---|---|---|
| Polglumyt ™ | 2 | — |
| Methyl p-hydroxybenzoate | 0.18 | 0.18 |
| Propyl p-hydroxybenzoate | 0.02 | 0.02 |
| Lanette O (cetylstearyl alcohol) | 5 | 5 |
| Lanette E (sodium cetylstearyl | 0.5 | 0.5 |
| sulfate) | | |
| Dimethicone 300 cps | 0.5 | 0.5 |
| Allantoin | 0.2 | 0.2 |
| Purified water q.s. | 100 | 100 |

### Example 3

Two face tonic formulations were prepared using the ingredients in Table 3 below. The quantities stated in Table 3 are percentages by weight (g/100 g).

TABLE 3

| | C1 (invention) | C2 (comparison) |
|---|---|---|
| Polglumyt ™ | 2 | — |
| Butylene glycol | 20 | 20 |
| Glycerine | 10 | 10 |
| PEG 60 | 2 | 2 |
| Hydrogenated castor oil | 2 | 2 |
| Sodium hyaluronate | 0.1 | 0.1 |
| Disodium EDTA | 0.1 | 0.1 |
| Phenoxyethanol | 0.5 | 0.5 |
| Methyl p-hydroxybenzoate | 0.12 | 0.12 |
| Perfume | 0.2 | 0.2 |
| Purified water q.s. | 100 | 100 |

### Example 4

The formulations in Examples 1 to 3 were subjected to evaluation according to the following procedure.

20 persons of the female sex aged between twenty and fifty years were selected. Instructions for the qualitative definition of perceived sensations specified in Table 4 below were provided to the persons included in the study.

TABLE 4

| Parameter | Description |
|---|---|
| Velvet sensation | Sensation of soft and smooth skin |
| Lifting sensation | Sensation of smooth and firm skin |
| Brightness sensation | Sensation of bright and lustrous skin |

The persons involved in the study were instructed to define the aforesaid perceptions quantitatively on the basis of the evaluation scale shown in Table 5 below.

TABLE 5

| Evaluation/Score | Description |
|---|---|
| None/(0) | Non-existent sensation |
| Low/(1) | Difficult to detect sensation |
| Medium/(2) | Easily detectable sensation |
| High/(3) | Very obvious sensation |

The persons involved in the study were also instructed to define the ease of absorption of the formulation under test in accordance with Table 6 below.

TABLE 6

| Evaluation/Score | Description |
|---|---|
| None/(0) | Time over 3 minutes |
| Low/(1) | Absorption within 3 minutes |
| Medium/(2) | Absorption within 2 minutes |
| High/(3) | Absorption within 1 minute |

Finally the persons included in the study were asked to provide an overall assessment of the product and the appearance of the product in accordance with Table 7 below.

TABLE 7

| Evaluation/Score | Description |
|---|---|
| 0 | Unacceptable |
| 1 | Acceptable |
| 2 | Good |
| 3 | Excellent |

In order to evaluate the sensory parameters of absorption and the overall assessments of the product and its appearance the assessments for the two formulations were compared using the signed-rank Wilcoxon statistical method.

Formulations A1, B1 and C1 containing Polglumyt™ glycogen formulations according to this invention were compared with the corresponding formulation A2, B2 and C2 without glycogen in accordance with a completely random cross double-blind experimental design. Three study sessions were carried out, one for each formulation. Each study session lasted one day. The formulations were applied by the individuals to the upper part of the forearm. The cream formulations were applied by hand spreading a quantity of approximately 3 grams. The tonic formulations were applied using a wad of cotton wool soaked in approximately 4 ml of solution. The area treated was massaged until the product was completely absorbed, for a time not exceeding three minutes.

The percent results and the value p of the statistical analysis of the evaluations made by the persons involved in the study for each parameter are summarised in the following tables. Table 8 relates to the results of the body cream products in Example 1. Table 9 relates to the results for the face cream products in Example 2. Table 10 relates to the results of the face tonic products in Example 3.

TABLE 8
BODY CREAM

| | | Evaluation % | | | | |
|---|---|---|---|---|---|---|
| Parameter | Product | 0 | 1 | 2 | 3 | Value of p |
| Velvet sensation | A1 | — | — | 40 | 60 | 0.074 |
| | A2 | — | 40 | 60 | — | |
| Lifting sensation | A1 | — | — | 40 | 60 | <0.0001 |
| | A2 | — | 40 | 60 | — | |
| Brightness sensation | A1 | — | — | 60 | 40 | NS |
| | A2 | — | 10 | 70 | 20 | |
| Absorption | A1 | — | — | 55 | 45 | 0.078 |
| | A2 | — | 15 | 70 | 15 | |
| Product | A1 | — | 10 | 30 | 60 | 0.0002 |
| | A2 | 10 | 50 | 35 | 5 | |
| Appearance | A1 | — | 30 | 25 | 45 | 0.0002 |
| | A2 | 15 | 75 | 10 | — | |

NS: Not significant

All the values (with the exception of the brightness sensation) showed a statistically significant difference in favour of the formulation according to this invention.

TABLE 9
FACE CREAM

| | | Evaluation % | | | | |
|---|---|---|---|---|---|---|
| Parameter | Product | 0 | 1 | 2 | 3 | Value of p |
| Velvet sensation | B1 | — | — | 45 | 55 | 0.0031 |
| | B2 | — | 50 | 35 | 15 | |
| Lifting sensation | B1 | — | — | 35 | 65 | 0.0015 |
| | B2 | — | 25 | 60 | 15 | |
| Brightness sensation | B1 | — | — | 40 | 60 | 0.0002 |
| | B2 | — | 40 | 50 | 10 | |
| Absorption | B1 | — | — | 50 | 50 | 0.0011 |
| | B2 | — | 50 | 40 | 10 | |
| Product | B1 | — | 20 | 45 | 35 | 0.0013 |
| | B2 | 20 | 50 | 30 | — | |
| Appearance | B1 | — | 55 | 40 | 5 | 0.0007 |
| | B2 | 50 | 45 | 5 | — | |

All the values showed a statistically significant difference in favour of the formulation according to this invention.

TABLE 10
FACE TONIC

| | | Evaluation % | | | | |
|---|---|---|---|---|---|---|
| Parameter | Product | 0 | 1 | 2 | 3 | Value of p |
| Velvet sensation | C1 | — | 5 | 40 | 55 | 0.0022 |
| | C2 | — | 60 | 35 | 5 | |
| Lifting sensation | C1 | — | 5 | 40 | 55 | 0.273 |
| | C2 | — | 60 | 35 | 5 | |
| Brightness sensation | C1 | — | 35 | 50 | 15 | NS |
| | C1 | — | 55 | 35 | 10 | |
| Absorption | C1 | — | 25 | 65 | 10 | NS |
| | C2 | — | 55 | 35 | 10 | |
| Product | C1 | 10 | 10 | 60 | 20 | NS |
| | C2 | 15 | 45 | 30 | 10 | |
| Appearance | C1 | 5 | 35 | 55 | 5 | 0.0074 |
| | C2 | 35 | 50 | 15 | — | |

NS: Not significant

The values for velvet and lifting sensation and the overall values relating to the appearance of the product showed a statistically significant difference in favour of the formulation according to this invention.

### Example 5

A lipstick formulation was prepared using the ingredients in Table 11 below. The quantities stated in Table 11 are percentages by weight (g/100 g).

TABLE 11

| Ingredient | Quantity |
|---|---|
| Polglumyt ™ | 2 |
| Vaseline oil | 28 |
| Microcrystalline wax | 13 |
| Paraffin | 13.4 |
| Hydrogenated coconut oil | 13 |
| Beeswax | 6 |
| Hydrogenated lanolin | 10 |
| Propyl p-hydroxybenzoate | 0.15 |
| Hydroxyanisol butylate (BHA) | 0.05 |
| Titanium dioxide | 1.20 |
| Zinc dioxide | 1.20 |
| UVA filter | 2 |
| UVB filter | 8 |
| Vitamin E acetate | 2 |

### Example 6

An aftershave formulation was prepared using the ingredients in Table 12 below. The quantities stated in Table 12 are percentages by weight (g/100 g).

TABLE 12

| Ingredient | Quantity |
|---|---|
| Polglumyt ™ | 2 |
| Water | 30 |
| Glycerol | 10 |
| Jojoba oil | 10 |
| Sorbitan oleate | 5 |
| Cetearyl olivate | 10 |
| Propylene glycol | 25 |
| Hydrogenated castor oil | 4 |
| Hydrogenated lecithin | 2.8 |
| Pantenol | 0.2 |
| Lactic acid | 0.1 |
| Tocopherol | 0.05 |
| Ascorbyl palmitate | 0.05 |
| Phenoxyethanol | 0.5 |
| Benzyl alcohol | 0.2 |
| Potassium sorbate | 0.1 |

### Example 7

A body foam formulation (mousse type) was prepared using the ingredients in Table 13 below. The quantities stated in Table 13 are percentages by weight (g/100 g).

TABLE 13

| Ingredient | Quantity |
|---|---|
| Polglumyt ™ | 2 |
| Caprylocaproyl polyoxylglycerides | 28 |
| (Caprylocaproyl | |
| macrogolglycerides) | |
| Propylene glycol monolaurate | 10 |
| Propylene glycol dicaprylocaproate | 2.5 |
| Hydrogenated castor oil | 15 |
| Methyl p-hydroxybenzoate | 0.12 |
| Water q.s. | 100 |

### Example 8

A hair lotion formulation was prepared using the ingredients in Table 14 below. The quantities stated in Table 14 are percentages by weight (g/100 g).

TABLE 14

| Ingredient | Quantity |
|---|---|
| Polglumyt ™ | 2 |
| Water | 58 |
| Glycerol | 10 |
| Olive oil esters | 6 |
| Sorbitan oleate | 5 |
| Cetearyl olivate | 3 |
| Betaine | 10 |
| Hydroxyethylcellulose | 4 |
| Pantenol | 1 |
| Lactic acid | 0.1 |
| Tocopherol | 0.05 |
| Ascorbyl palmitate | 0.05 |
| Phenoxyethanol | 0.5 |
| Benzyl alcohol | 0.2 |
| Potassium sorbate | 0.1 |

### Example 9

A sun oil formulation was prepared using the ingredients in Table 15 below. The quantities stated in Table 15 are percentages by weight (g/100 g).

TABLE 15

| Ingredient | Quantity |
|---|---|
| Polglumyt ™ | 2 |
| Caprylic and capric acid esters | 20 |
| (Cetiol ™ LC) | |
| Caprylic and capric acid | 40 |
| triglyceride | |
| (Myritol ™ 318) | |
| 2-octyldodecanol | 36 |
| UV filter | 2 |

1. A cosmetic composition for skin application having a velvet effect on the skin comprising glycogen containing less than 1,000 ppm of nitrogen and less than 1% by weight of reducing sugars, in at least one cosmetically-acceptable vehicle.

2. The cosmetic composition according to claim 1, wherein said glycogen comprises less than 100 ppm of nitrogen and less than 0.25% by weight of reducing sugars.

3. The cosmetic composition according to claim 1, wherein said composition comprises a quantity of said glycogen of between about 0.1% and about 15% by weight relative to the weight of the total formulation.

4. The cosmetic composition according to claim 3, wherein said composition comprises a quantity of said glycogen of between 0.5% and 10% by weight relative to the weight of the total formulation.

5. The cosmetic composition according to claim 3, wherein said composition comprises a quantity of said glycogen of between 1% and 5% by weight relative to the weight of the total formulation.

6. The cosmetic composition according to claim 1, wherein said cosmetically-acceptable vehicle is selected from the group consisting of emulsifiers, hydrating agents, solvents, emollients, stabilisers, viscosity agents, preservatives, lubricants, sequestrating or chelating agents, filters, fillers, fragrances, perfumes, absorbants, absorbents, colouring agents and opacifiers, antioxidants, plant extracts and oils, vitamins, foaming protective substances, essential oils, keratin-active substances and amino acids.

7. The cosmetic composition according to claim 1, wherein said composition comprises liquid or semi-solid formulations.

8. The cosmetic composition according to claim 7, wherein said liquid formulation comprises at least one solvent, at least one hydrating agent, at least one sequestering agent, and at least one preservative.

9. The cosmetic composition according to claim 7, wherein said semi-solid formulation comprises at least one solvent, at least one emulsifier, at least one viscosity agent, at least one hydrating agent, at least one emollient, and at least one preservative.

10. The cosmetic composition according to claim 6, wherein said solvent is selected from the group consisting of water, alcohols, ketones, glycols, polyethylene glycols, alkyl acetates, isoparaffins, cycloalkyls, glycerine, natural and synthetic oils, natural and synthetic triglycerides.

11. The cosmetic composition according to claim 6, wherein said emulsifier is selected from the group consisting of sorbitans, ethoxylated long-chain alcohols, alkyl polyglycosides, soaps, alkyl sulphates, sulfates, monoalkyl and dialkyl phosphates, alkyl sulphonates, sulfonates, hydrogenated castor oil, acyl isothionates, saccharose esters, betaine, lecithin, quaternary ammonium salts, alkyl oleates, glycerides and olive oil emulsifiers.

12. The cosmetic composition according to claim 6, wherein said viscosity agent is selected from the group consisting of xanthan gum, hydroxypropyl cellulose, hydroxyethyl cellulose, carbopol, carragheenans, poloxymerscarrageenans, poloxamers and acacia gums.

13. The cosmetic composition according to claim 6, wherein said hydrating agent is selected from the group consisting of urea, allantoin, hyaluronic acid and its derivatives, glycerine, amino acids, acetyl monoethanolamide, butoxypropanol, butyl glycol, low molecular weight polyethylene glycols, aloe, mallow and sorbitol.

14. The cosmetic composition according to claim 6, wherein said emollient is selected from the group consisting of lanolin, almond oil, olive oil, hydrogenated castor oil, microcrystalline wax, polydimethylsiloxane (dimethicone), polymethylphenylsiloxane, polymers of glycol and silicone, mineral oils, paraffin, ozokerite, ceresine, triglyceride esters, monoglyceride acetylates, glyceride ethoxylates, ethoxylated glycerides, alkyl esters of fatty acids, fatty acids, long-chain alcohols, sterols, beeswax, polyhydric alcohols, polyesters, and fatty acid amides.

15. The cosmetic composition according to claim 6, wherein said preservative is selected from the group consisting of alcohols, methyl and propyl parahydroxy benzoate, hydroxyanisole butylate (BHA), sorbates, urea derivates, and isothiazolinones.

16. The cosmetic composition according to claim 6, wherein said sequestering agent is selected from the group consisting of a EDTA, HEDTA, alkyl oxalates, lithium or potassium oxalate, and sodium or potassium pyrophosphate.

17. The cosmetic composition according to claim 6, wherein said composition comprises a quantity of water up to 99% by weight relative to the weight of the total formulation.

18. The cosmetic composition according to claim 17, wherein said composition comprises a quantity of water of between 25% and 95% by weight relative to the weight of the total formulation.

19. The cosmetic composition according to claim 17, wherein said composition comprises a total quantity of non-aqueous solvents of between approximately about 0.1% and approximately about 60% by weight relative to the weight of the total formulation.

20. The cosmetic composition according to claim 6, wherein said composition comprises a quantity of water of less than 0.01% by weight relative to the weight of the total formulation and a total quantity of non-aqueous solvents of between about 1% and about 99% by weight relative to the weight of the total formulation.

21. The cosmetic composition according to claim 6, wherein said composition comprises a quantity of said emulsifier of between about 0.1% and about 60% by weight relative to the weight of the total formulation.

22. The cosmetic composition according to claim 6, wherein said composition comprises a quantity of said viscosity agent of between about 0.1% and about 25% by weight relative to the weight of the total formulation.

23. The cosmetic composition according to claim 6, wherein said composition comprises a quantity of said hydrating agent of between about 0.05% and about 25% by weight relative to the weight of the total formulation.

24. The cosmetic composition according to claim 6, wherein said composition comprises a quantity of said emollient agent of between about 0.1% and about 25% by weight relative to the weight of the total formulation.

25. The cosmetic composition according to claim 6, wherein said composition comprises a quantity of said preservative of between about 0.01% and about 2.00% by weight relative to the weight of the total formulation.

26. The cosmetic composition according to claim 6, wherein said composition comprises a quantity of said sequestering agent of between about 0.01% and about 20% by weight relative to the weight of the total formulation.

27-29. (canceled)

30. A method for the preparation of a cosmetic composition for skin application which provides a velvet effect on the skin, said method comprising incorporating into said composition a glycogen according to claim 1.

31. A method of obtaining a velvet effect on the skin, said method comprising applying to the skin a cosmetic composition comprising glycogen according to claim 1.

32. A method of obtaining a velvet effect on the skin by using a cosmetic composition for skin application which comprises a glycogen according to claim 1.
